(19) United States
(12) Patent Application Publication
(10) Pub. No.: US 2024/0306998 A1
(43) Pub. Date: Sep. 19, 2024

(54) METHODS AND SYSTEMS FOR TRAINING EEG SIGNAL DENOISING MODELS OF EEG COLLECTION DEVICES

(71) Applicant: SICHUAN NEOSOURCE BIOTEKTRONICS LIMITED, Chengdu (CN)

(72) Inventors: Jiawei ZHANG, Chengdu (CN); Chao CHEN, Chengdu (CN)

(73) Assignee: SICHUAN NEOSOURCE BIOTEKTRONICS LIMITED, Chengdu (CN)

(21) Appl. No.: 18/673,237

(22) Filed: May 23, 2024

Related U.S. Application Data
(63) Continuation of application No. PCT/CN2022/137825, filed on Dec. 9, 2022.

(30) Foreign Application Priority Data
Dec. 9, 2021 (CN) 202111503309.9

(51) Int. Cl. A61B 5/00; A61B 5/291; A61B 5/369

(52) U.S. Cl. CPC A61B 5/7203; A61B 5/369; A61B 5/7267; A61B 5/7278; A61B 5/291; A61B 2560/045

(57) ABSTRACT

A method and system for training an EEG signal denoising model of an EEG collection device is provided. The method includes generating an analog EEG signal and obtaining a collected EEG signal by collecting the analog EEG signal through the EEG collection device. The collected EEG signal contains a noise caused by the EEG collection device. The method further includes obtaining the EEG signal denoising model of the EEG collection device by training a denoising model based on the collected EEG signal. The training includes obtaining a denoised EEG signal by processing the collected EEG signal through the denoising model; obtaining a discrimination result by processing the denoised EEG signal through a discriminant model; and adjusting a model parameter of the denoising model based on the discrimination result. The discrimination result reflects a probability that the discriminant model determines that the processed denoised EEG signal is a clean EEG signal.

### CROSS-REFERENCE TO RELATED APPLICATION

This application is a continuation of International Application No. PCT/CN2022/137825, filed on Dec. 9, 2022, which claims priority to Chinese application No. 202111503309.9, filed on Dec. 9, 2021, the entire content of each of which is incorporated herein by reference.

### TECHNICAL FIELD

The present disclosure relates to a field of electroencephalography (EEG) signal processing, and in particular to, a method and system for training an EEG signal denoising model of an EEG collection device.

### BACKGROUND

An EEG signal (e.g., an electroencephalogram) refers to a signal generated by amplifying and recording weak bioelectricity of a human brain, which may be collected by an EEG collection device. The EEG signal is widely used in research in directions of psychology, neuroscience, psychiatry, and brain-computer interfaces.

The EEG signal has features of diversity, nonlinearity, and weakness, and frequency bands of the EEG signal are mainly in low-frequency and ultra-low-frequency ranges. For example, a main frequency of the EEG signal is in a range between 0.5 and 100 Hz, and a signal amplitude of the EEG signal is in a range of 5 and 300 μV. As the EEG signal is quite weak, it is easily interfered with and drowned out by noise, such as the noise brought by the EEG collection device itself during the collection process, as well as other noises, such as environmental noise, an artifact, etc. So, in order to collect a clean EEG signal, it is necessary to denoise a brainwave signal collected by the EEG collection device, in particular to remove the noise that is brought by the EEG collection device itself during the collection process.

Therefore, there is a need for a method and system for training an EEG signal denoising model of the EEG collection device, to train and obtain a denoising model of the EEG signal for removing the noise in the EEG signal collected by the EEG collection device, especially the noise brought by the EEG collection device itself during the collection process.

### SUMMARY

One of the embodiments of the present disclosure provides a method implemented on a computing device. The computing device includes at least one processor and at least one storage medium storing a training instruction set for training an EEG signal denoising model of an EEG collection device. The method includes generating an analog EEG signal; obtaining a collected EEG signal by collecting the analog EEG signal through the EEG collection device, the collected EEG signal containing a noise caused by the EEG collection device; and obtaining the EEG signal denoising model of the EEG collection device by training a denoising model based on the collected EEG signal. The training of the denoising model includes obtaining a denoised EEG signal by processing the collected EEG signal through the denoising model; obtaining a discrimination result by processing the denoised EEG signal through a discriminant model, the discrimination result reflecting a probability that the discriminant model determines that the processed denoised EEG signal is a clean EEG signal; and adjusting a model parameter of the denoising model based on the discrimination result.

One of the embodiments of the present disclosure provides a system including at least one storage medium storing a training instruction set for an EEG signal denoising model of an EEG collection device and at least one processor in communication with the at least one storage medium. When performing the instruction set, the at least one processor is configured to generate an analog EEG signal; obtain a collected EEG signal by collecting the analog EEG signal through the EEG collection device, the collected EEG signal containing a noise caused by the EEG collection device; and obtain the EEG signal denoising model of the EEG collection device by training a denoising model based on the collected EEG signal. The training of the denoising model includes obtaining a denoised EEG signal by processing the collected EEG signal through the denoising model; obtaining a discrimination result by processing the denoised EEG signal through a discriminant model, the discrimination result reflecting a probability that the discriminant model determines that the processed denoised EEG signal is a clean EEG signal; and adjusting a model parameter of the denoising model based on the discrimination result.

One of the embodiments of the present disclosure provides a training device for an EEG signal denoising model of an EEG collection device, including at least one storage medium configured to store computer instructions and at least one processor configured to perform the computer instructions to implement the method of: generating an analog EEG signal; obtaining a collected EEG signal by collecting the analog EEG signal through the EEG collection device, the collected EEG signal containing a noise caused by the EEG collection device; and obtaining the EEG signal denoising model of the EEG collection device by training a denoising model based on the collected EEG signal. The training of the denoising model includes obtaining a denoised EEG signal by processing the collected EEG signal through the denoising model; obtaining a discrimination result by processing the denoised EEG signal through a discriminant model, the discrimination result reflecting a probability that the discriminant model determines that the processed denoised EEG signal is a clean EEG signal; and adjusting a model parameter of the denoising model based on the discrimination result.

One of the embodiments of the present disclosure provides a computer-readable storage medium storing computer instructions. When reading the computer instructions, a computer implements a method including generating an analog EEG signal; obtaining a collected EEG signal by collecting the analog EEG signal through the EEG collection device, the collected EEG signal containing a noise caused by the EEG collection device; and obtaining the EEG signal denoising model of the EEG collection device by training a denoising model based on the collected EEG signal. The training of the denoising model includes obtaining a denoised EEG signal by processing the collected EEG signal through the denoising model; obtaining a discrimination result by processing the denoised EEG signal through a discriminant model, the discrimination result reflecting a probability that the discriminant model determines that the processed denoised EEG signal is a clean EEG signal; and adjusting a model parameter of the denoising model based on the discrimination result.

### BRIEF DESCRIPTION OF THE DRAWINGS

The present disclosure is further illustrated by way of exemplary embodiments, which are described in detail by means of the accompanying drawings. These embodiments are not limiting, and in these embodiments, the same numbering denotes the same structure, wherein:

FIG. 1 is a schematic diagram illustrating an application scenario of a method for training an EEG signal denoising model of an EEG collection device according to some embodiments of the present disclosure;

FIG. 2 is a schematic diagram illustrating hardware and/or software assemblies of an exemplary computing device according to some embodiments of the present disclosure;

FIG. 3 is a schematic diagram illustrating hardware and/or software assemblies of an exemplary mobile device according to some embodiments of the present disclosure;

FIG. 4 is a flowchart illustrating an exemplary method for training an EEG signal denoising model of an EEG collection device according to some embodiments of the present disclosure;

FIG. 5 is a flowchart illustrating training of a denoising model of an EEG signal denoising model of an EEG collection device according to some embodiments of the present disclosure;

FIG. 6 is a schematic diagram illustrating a method for training a denoising model in a staged manner according to some embodiments of the present disclosure;

FIG. 7 is a flowchart illustrating an exemplary process for adjusting a model parameter of a denoising model according to some embodiments of the present disclosure; and

FIG. 8 is a module diagram illustrating a system for training an EEG signal denoising model of an EEG collection device according to some embodiments of the present disclosure.

### DETAILED DESCRIPTION

In order to more clearly illustrate the technical solutions of the embodiments of the present disclosure, the accompanying drawings required to be used in the description of the embodiments are briefly described below. Obviously, the accompanying drawings in the following description are only some examples or embodiments of the present disclosure, and it is possible for those skilled in the art to apply the present disclosure to other similar scenarios in accordance with these drawings without creative labor. Unless obviously obtained from the context or the context illustrates otherwise, the same numeral in the drawings refers to the same structure or step.

It may be understood that the terms “system,” “device,” “unit,” and/or “module” as used herein are a way to distinguish between different components, elements, parts, sections, or assemblies at different levels. However, the words may be replaced by other expressions if other words accomplish the same purpose.

As shown in the present disclosure and the claims, unless the context clearly suggests an exception, the words “a,” “one,” “an,” and/or “the” do not refer specifically to the singular, but may also include the plural. Generally, the terms “including” and “comprising” suggest only the inclusion of clearly identified steps and elements. In general, the terms “including” and “comprising” only suggest the inclusion of explicitly identified steps and elements that do not constitute an exclusive list, and the method or apparatus may also include other steps or elements.

Flowcharts are used in the present disclosure to illustrate steps performed by a system in accordance with embodiments of the present disclosure. It may be appreciated that the preceding or following steps are not necessarily performed in an exact sequence. Instead, the steps may be processed in reverse order or simultaneously. Also, it may be possible to add other steps to these processes or remove a step or steps from them.

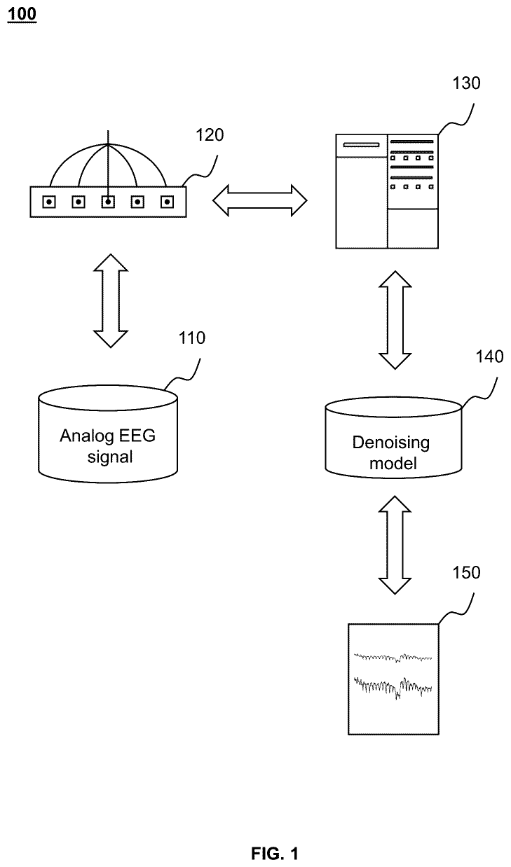

FIG. 1 is a schematic diagram illustrating an application scenario of a method for training an EEG signal denoising model of an EEG collection device according to some embodiments of the present disclosure.

The method for training the EEG signal denoising model of the EEG collection device may be used for training a denoising model to obtain the EEG signal denoising model capable of performing denoising on an EEG signal collected by the EEG collection device. As shown in FIG. 1, an application scenario 100 of the method for training the EEG signal denoising model of the EEG collection device may include an analog EEG signal 110 generated by an EEG signal generator, an EEG collection device 120, and a processing device 130.

The EEG signal generator may include a variety of signal generators capable of generating the analog EEG signal. In some embodiments, the EEG signal generator may include a multi-channel EEG signal generator to simulate a multi-channel EEG signal collected from different brain portions.

The EEG collection device 120 may obtain a collected EEG signal by collecting the analog EEG signal. The EEG collection device 120 may be connected to the processing device 130 (e.g., via a network connection or a direct connection) to enable the processing device 130 to access and process the collected EEG signal. The EEG collection device 120 refers to a device for collecting the EEG signal. For example, the EEG collection device may include an EEG meter, an EEG monitor, a portable EEG collection device, etc. The EEG collection device 120 may include a plurality of electrode slices used for collecting the EEG signal. For example, the electrode slices may be connected to a signal output end of the EEG signal generator to collect the analog EEG signal, or be fitted to various portions of a human brain to collect the EEG signal of the human brain.

The processing device 130 may process various data or information, perform calculations, and determine various results. In some embodiments, the processing device 130 may process the collected EEG signal obtained by the EEG collection device 120. For example, the processing device 130 may train a denoising model 140 based on the collected EEG signal of the EEG collection device to obtain a corresponding EEG signal denoising model corresponding to the EEG collection device. The processing device 130 may process data and/or information obtained from other devices or system components. The processor may perform program instructions based on such data, information, and/or processing results to perform one or more of the functions described in the present disclosure. In some embodiments, the processing device 130 may include one or more sub-processing devices (e.g., a single-core processing device, a multi-core processing device, etc.). In some embodiments, the EEG signal denoising model may also be referred to as a denoising model.

The processing device 130 may process the collected EEG signal (e.g., the EEG signal collected on the human brain) obtained by the EEG collection device 120 through an EEG signal denoising model to obtain a corresponding denoised EEG signal 150 (e.g., a clean EEG signal).

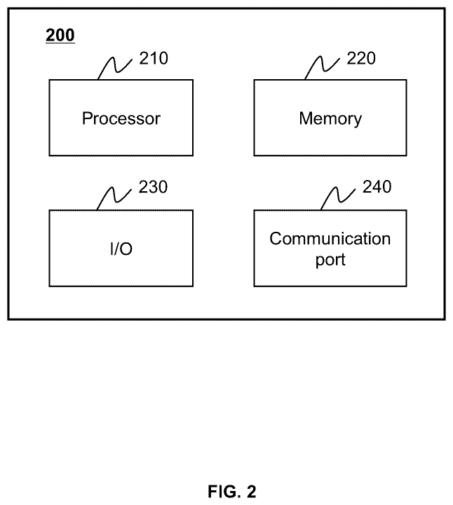

FIG. 2 is a schematic diagram illustrating hardware and/or software assemblies of an exemplary computing device according to some embodiments of the present disclosure. As shown in FIG. 2, a computing device 200 may include a processor 210, a memory 220, an input/output (I/O) 230, and a communication port 240. In some embodiments, the processing device 130 may be implemented on the computing device 200.

The processor 210 may perform computer instructions (program codes). When performing the computer instructions, the processor 210 may cause the processing device 130 to perform the functions of the processing device 130 according to the techniques described herein. The computer instructions may include, for example, routines, programs, objects, components, signals, data structures, processes, modules, and functions that perform particular functions described herein. In some embodiments, the processor 210 may process data and/or images obtained from the EEG collection device 120, the processing device 130, and/or any other components of a system for training the EEG signal denoising model of the EEG collection device. In some embodiments, the processor 210 may include one or more hardware processors, such as a microcontroller, microprocessor, a reduced instruction set computer (RISC), an application specific integrated circuit (ASIC), an application specific instruction set processor (ASIP), a central processing unit (CPU), a graphics processing unit (GPU), a physical processing unit (PPU), a microcontroller unit, a digital signal processor (DSP), a field programmable gate array (FPGA), an advanced RISC machine (ARM), a programmable logic device (PLD), any circuitry or processor capable of performing one or more functions, etc., or any combination thereof.

For illustrative purposes only, only one processor is described in computing device 200. However, it may be noted that the computing device 200 of the present disclosure may also include multiple processors. Accordingly, operations, methods, and/or steps performed by a single processor as described in the present disclosure may also be performed by the multiple processors, either jointly or separately. For example, if both process A and process B are performed in the processor of the computing device 200 of the present disclosure, it may be understood that the process A and the process B may also be performed in the computing device 200 by two or more different processors, either together or separately (e.g., a first processor performs the process A and a second processor performs the process B, or a first processor and a second processor jointly perform the processes A and B).

The memory 220 may store data/information obtained from the EEG collection device 120, the processing device 130, and/or any other components of the system for training the EEG signal denoising model of the EEG collection device. In some embodiments, the memory 220 may include a mass storage device, a removable storage device, a volatile read and write memory, a read-only memory (ROM), etc., or any combination thereof. For example, the mass storage device may include a disk, a CD-ROM, a solid state drive, etc. The removable storage device may include a flash drive, a floppy disk, an optical disk, a memory card, a zip disk, a magnetic tape, etc. The volatile read and write memory may include a random access memory (RAM). The RAM may include a dynamic RAM (DRAM), a double rate synchronous dynamic RAM (DDR SDRAM), a static RAM (SRAM), a thyristor RAM (T-RAM), and a zero capacitance (Z-RAM). The ROM may include a masked ROM (MROM), a programmable ROM (PROM), an erasable programmable ROM (PEROM), an electrically erasable programmable ROM (EEPROM), a compact disk ROM (CD-ROM), and a digital universal disk ROM. In some embodiments, the memory 220 may store one or more programs and/or instructions to perform the exemplary methods described in the present disclosure. For example, the memory 220 may store a program (e.g., in a form of computer-executable instructions) for processing device 130 for determining a calibration model for calibration. As another example, the memory 220 may store a program (e.g., in a form of computer-executable instruction) for the processing device 130 for determining whether the calibration model needs to be updated.

The I/O 230 may input or output a signal, data, and/or information. In some embodiments, the I/O 230 may enable a user to interact with the processing device 130. In some embodiments, the I/O 230 may include an input device and an output device. An exemplary input device may include a keyboard, a mouse, a touch screen, a microphone, etc., or any combination thereof. An exemplary output device may include a display device, a speaker, a printer, a projector, etc., or any combination thereof. An exemplary display device may include a liquid crystal display (LCD), a light emitting diode (LED) based display, a flat panel display, a curved display, a television device, a cathode ray tube (CRT), etc., or any combination thereof.

The communication port 240 may be connected to a network to facilitate data communication. The communication port 240 may establish a connection between the processing device 130 and the EEG collection device 120. The connection may be a wired connection, a wireless connection, or a combination thereof for data transmission and reception. The wired connection may include a cable connection, a fiber optic cable connection, a telephone line connection, etc., or any combination thereof. The wireless connection may include a Bluetooth connection, a Wi-Fi connection, a WiMAX connection, a WLAN connection, a ZigBee connection, a mobile network (e.g., 3G, 4G, 5G, etc.) connection, or a combination thereof. In some embodiments, the communication port 240 may be a standardized port such as RS232, RS485, etc. In some embodiments, the communication port 240 may be a specially designed communication port. For example, the communication port 240 may be designed according to digital imaging and communications in medicine (DICOM) protocol.

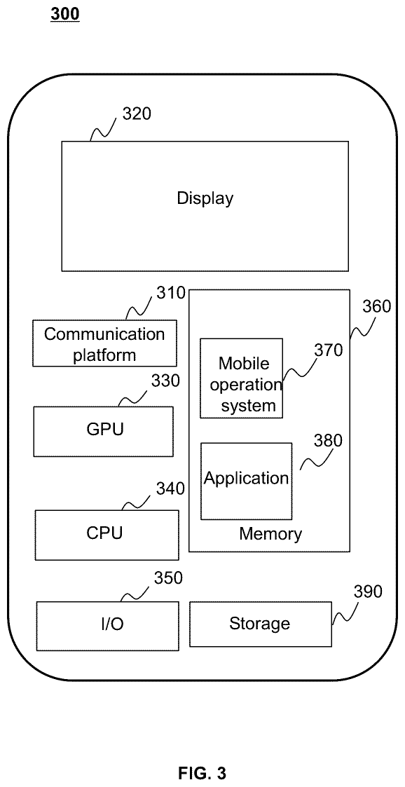

FIG. 3 is a schematic diagram illustrating hardware and/or software assemblies of an exemplary mobile device according to some embodiments of the present disclosure. In some embodiments, the processing device 130 may be implemented on the computing device 200. As shown in FIG. 3, a mobile device 300 may include a communication platform 310, a display 320, a graphics processing unit (GPU) 330, a central processing unit (CPU) 340, an I/O 350, a memory 360, and a storage 390. In some embodiments, any other suitable assemblies, including, but not limited to, a system bus or a controller (not shown), may also be included in the mobile device 300. In some embodiments, a mobile operation system 370 (e.g., IOS, Android, Windows Phone, etc.) and one or more applications 380 may be loaded from the storage 390 into the memory 360 for execution by the CPU 340. The applications 380 may include a browser or any other suitable mobile applications for receiving and rendering information related to image processing or other information from the processing device 130. An interaction between a user and an information flow may be realized through the I/O 350 and provided to, via a network, the processing device 130 and/or other components of the system for training the EEG signal denoising model of the EEG collection device.

To implement the various modules, units, and functions thereof described herein, a computer hardware platform may be used as a hardware platform for one or more of the assemblies described herein. Hardware elements, operating systems, and programming languages for various computers are practically regular and are assumed to be sufficiently familiar to those skilled in the art to make the techniques suitable for generating high-quality images for the scanned objects described herein. The computer containing user interface elements may be used as a personal computer (PC) or other type of workstation or terminal device, or a server when properly programmed. It may be understood that those skilled in the art should be familiar with the structure, programming, and general step of such a computer device, and thus the figures should be self-explanatory thereof.

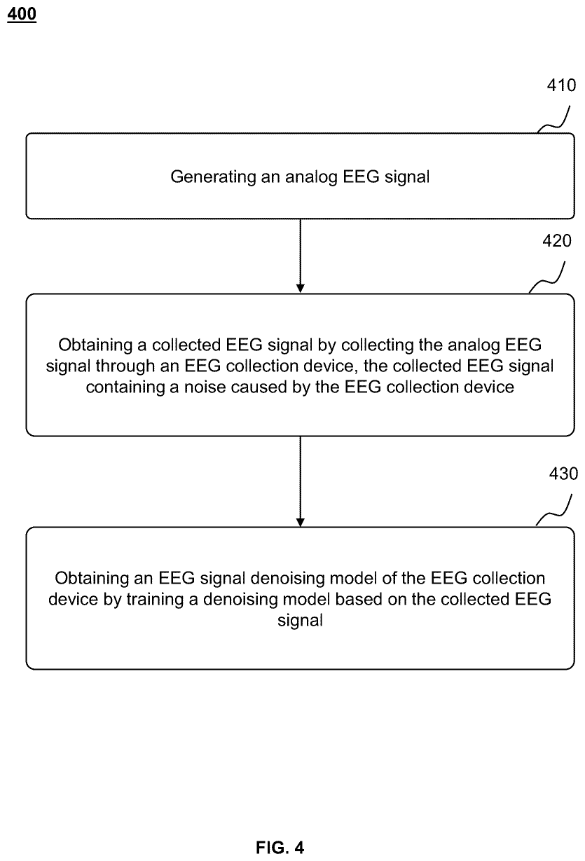

FIG. 4 is a flowchart illustrating an exemplary method for training an EEG signal denoising model of an EEG collection device according to some embodiments of the present disclosure. As shown in FIG. 4, a process 400 includes the following steps.

In step 410, an analog EEG signal is generated. In some embodiments, step 410 may be performed by a generation module 810.

The analog EEG signal refers to a signal that simulates an EEG signal of a human brain. In some embodiments, a processing device may obtain a digital signal by various analog EEG signal generation manners, and then cause an EEG signal generator to convert the digital signal to the analog signal output. For example, the analog EEG signal generation manner may include an EEG signal synthesis algorithm, a generation model for generating the analog EEG signal, a generation network, an encoder, etc.

In some embodiments, the analog EEG signal may include a clean analog EEG signal. The analog EEG signal may include a first analog EEG signal reflecting an underlying feature of the EEG signal, and may further include a second analog EEG signal simulating a clean EEG signal. In some embodiments, the analog EEG signal may further include an analog EEG signal with noise. Specifically, the analog EEG signal may include a third analog EEG signal with at least one other noise superimposed on the clean EEG signal.

The underlying feature of the EEG signal may include signal underlying features such as an amplitude feature (e.g., an amplitude range), a frequency feature (e.g., a frequency range), etc. The first analog EEG signal may have a wave amplitude that changes regularly and standardly, which is capable of reflecting the underlying features of the EEG signal. For example, the first analog EEG signal may include a sinusoidal signal reflecting the underlying features of the EEG signal.

The second analog EEG signal may be an analog EEG signal that is more complex relative to the first analog EEG signal. A change rule of the second analog EEG signal may be more in line with the change rule of a real EEG signal.

In some embodiments, the second analog EEG signal may be generated by a generation model. In some embodiments, the processing device may input arbitrary data (e.g., random white noise) into the generation model, and the generation model may output the second analog EEG signal.

In some embodiments, the processing device may train the generation model jointly with a discriminant model. The processing device may combine a discriminant model and a generation model to obtain a generative adversarial network (GAN) and train the GAN to obtain the generation model. The generation model may be a generator in the GAN, and the discriminant model may be a discriminator in the GAN. In some embodiments, the generation model may include, but is not limited to, a variety of models such as an NN, an RNN, a CNN, etc. that can generate the analog EEG signal. In some embodiments, the discriminant model may determine whether the input data is the real EEG signal or the analog EEG signal. The discriminant model may include, but is not limited to, a decision tree, an NN, a KNN, etc. that can used to discriminate a type of the input data. In some embodiments, the training of the generation model may include training the generation model based on the discriminant model (which is referred to as a generation model training stage). At this time, the discriminant model may be a trained discriminant model. In the generation model training stage, the processing device may fix a parameter of the discriminant model, input the second analog EEG signal generated by the generation model into the discriminant model, and adjust the parameter of the generation model according to the output of the discriminant model. A training label may be that the discrimination result output by the discrimination model is real, i.e., the discriminant model determines the second analog EEG signal output by the generation model as the real EEG signal.

In some embodiments, the training of the generation model may also include training the discriminant model (which is referred to as the discriminant model training stage). In the discriminant model training stage, the parameter of the generation model may be fixed to train the discriminant model. The processing device may generate a plurality of second analog EEG signals based on the generation model, and select a plurality of real EEG signals from historical data, and then designate the plurality of second analog EEG signals and the plurality of real EEG signals as a training sample. The data types corresponding to the second analog EEG signals and the real EEG signals as the labels to train the discriminant model. As a result, the discriminant model may distinguish between the second analog EEG signals and the real EEG signals as much as possible. In some embodiments, the processing device may alternately perform the discriminant model training stage and the generation model training stage during the training. Through a plurality of iterations of the training, the generation model and the discriminant model may become more and more capable, and when the generation model satisfies conditions such as model convergence, the trained generation model may be obtained. In some embodiments, during the training process of the generation model described above, the data types corresponding to the second analog EEG signal and the real EEG signal in the training samples may be automatically labeled according to the different data sources, or may be manually labeled.

The noise refers to a signal that interferes with the EEG signal. In the present disclosure, other noise refers to the noise other than the noise brought by the EEG signal collection device. The noise may include an artifact signal generated by a blinking of the human body, a heart rate, and a muscle movement of the human body in the human body's EEG signals, and it may also include environmental noise, etc.

In some embodiments, the processing device may generate a third analog EEG signal by superimposing at least one other noise on a clean EEG signal. The processing device may superimpose at least one other noise on a clean real EEG signal (e.g., the denoised EEG signal output from the denoising model after a collected real EEG signal is input into the denoising model) or a clean analog EEG signal (e.g., the second analog EEG signal) to generate the third analog EEG signal. For example, the processing device may superimpose an artificially generated environmental noise signal and the artifact generated by the human heart rate on the clean real EEG signal to generate the third analog EEG signal.

In some embodiments, by generating different third analog EEG signals by superimposing different other noises on the same clean EEG signal, large-scale training data may be obtained based on a small number of clean EEG signals, which solves the problem of small training data for the denoising model.

In step 420, a collected EEG signal is obtained by collecting the analog EEG signal through the EEG collection device, the collected EEG signal containing a noise caused by the EEG collection device. In some embodiments, step 420 may be performed by a collection module 820.

The EEG collection device may collect the EEG signal of the human brain and the analog EEG signal of the EEG signal generator to obtain the corresponding collected EEG signal. For the EEG collection device, as its electronic components need a power supply to operate, situations like a power supply jitter, a temperature drift of the components, a possible tampering of internal lines of each channel, and changes of an external electromagnetic environment usually occur in an operation of the EEG collection device. All of these situations may generate the noise signals during the EEG collection and recording process, which interfere with the EEG signal, e.g., reduce a signal-to-noise ratio of the EEG signal. The noise generated by the operation of the EEG collection device itself is the noise brought about by the EEG collection device. For example, when the third analog EEG signal is the EEG signal with an artificially generated environmental noise superimposed on the clean real EEG signal and the EEG collection device collects the third analog EEG signal, the EEG signal collected by the EEG collection device may have both the original environmental noise and the noise caused by the EEG collection device.

In step 430, an EEG signal denoising model of the EEG collection device is obtained by training a denoising model based on the collected EEG signal. In some embodiments, step 430 may be performed by a training module 830.

The denoising model refers to a model that removes the noise from the input collected EEG signal and outputs a denoised EEG signal (e.g., the clean EEG signal). In some embodiments, the denoising model may include, but is not limited to, various models such as an NN, an RNN, a CNN, etc., that are capable of performing the denoising processing on the EEG signal.

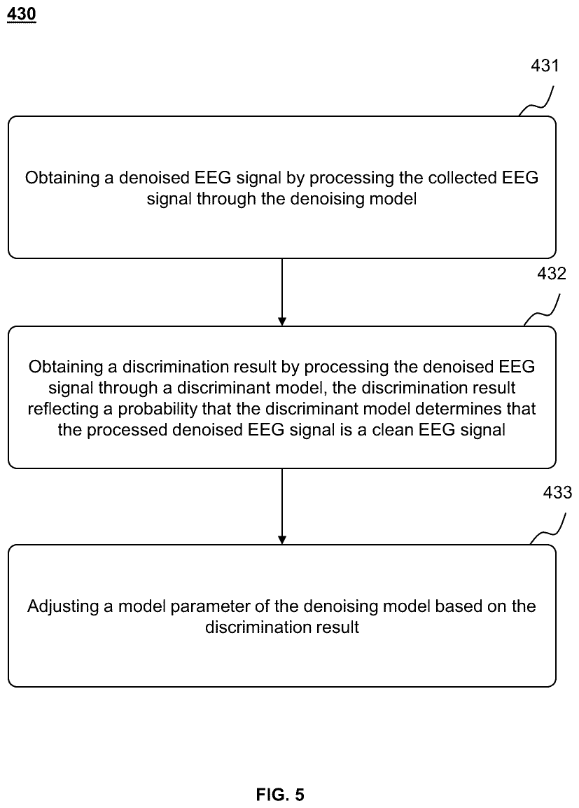

In some embodiments, the processing device may train the denoising model in conjunction with the discriminant model to obtain a desired denoising model of the EEG signal corresponding to the EEG collection device. A main role of the discriminant model is to make the denoised signal output by the denoising model as clean an EEG signal as possible. It may be noted that the discriminant model that is joint during the training of the denoising model and the discriminant model that is joint during the training of the generation model that generates the second analog EEG signal are different models, although their model structures may be the same. Referring to FIG. 5, in some embodiments, the training of the denoising model may include the following steps.

In step 431, a denoised EEG signal is obtained by processing the collected EEG signal through the denoising model.

The processing device may input the collected EEG signal into the denoising model for the denoising processing, and the output of the denoising model may be referred to as the denoised EEG signal.

In step 432, a discrimination result is obtained by processing the denoised EEG signal through a discriminant model, the discrimination result reflecting a probability that the discriminant model determines that the processed denoised EEG signal is a clean EEG signal.

The discrimination result output by the discriminant model may be expressed as a probability or a score. For example, 1 indicates the data being processed (i.e., the input data) (e.g., the denoised EEG signal output by the denoising model) is a clean EEG signal; 0 indicates that the data being processed (i.e., the input data) is not a clean EEG signal, i.e., the input data is the EEG signal containing the noise. The higher the probability or score of the discriminant model output, the higher the probability that the corresponding input data is the clean EEG signal.

In some embodiments, the discriminant model may be a multiclassification model. A plurality of types corresponding to the multiclassification may include the EEG signals with various types of noises (e.g., the EEG signal with eye movement artifacts, the EEG signal with muscle movement artifacts, the EEG signal with environmental noise, etc.) and the clean EEG signal. The discrimination result may also reflect a probability of discriminating the data being processed as the EEG signals with various types of noises. In such cases, the discrimination result may include a probability or score that the denoised EEG signal belongs to each of the plurality of types. For example, the denoised EEG signal obtained by the denoising model may be input into the discriminant model, and the discrimination result obtained by the discriminant model (which is also referred to as a multiclassification result) may include a probability that the denoised EEG signal belongs to a plurality of types. For example, a probability for the denoising EEG signal obtained by the discriminant model to belong to the EEG signal with an eye movement artifact may be 0.2, a probability for the EEG signal to belong to the EEG signal with a muscle movement artifact may be 0.2, a probability for the denoised EEG signal to belong to the EEG signal with the environmental noise may be 0.3, and a probability for the denoised EEG signal to belong to the clean EEG signal may be 0.6. The processing device may take the type with the highest probability as a classification result of the denoised EEG signal.

It can be understood that during the training, the denoising capability of the denoising model changes from weak to strong. When the denoising capability of the denoising model is weak, there may still be the noise in the denoised signal output from the denoising model, so the output of the discriminant model may vary with the capability of the denoising model during the training process.

In step 433, a model parameter of the denoising model is adjusted based on the discrimination result.

When training the denoising model, the processing device may fix the parameter of the discriminant model (in such cases, the discriminant model may be regarded as the trained discriminant model). A target of the adjusting the model parameter of the denoising model by training may include making the denoising model to remove as much noise as possible from the collected EEG signal and output an as clean as possible denoised EEG signal. As a result, after inputting the denoised EEG signal into the discriminant model, the obtained discriminant result may indicate that a probability of the input data being a clean EEG signal is relatively great or is 1.

In some embodiments, as previously described, the discriminant model may be the multiclassification model, and the plurality of types corresponding to the multiclassification may include the EEG signals with noises of various types and the clean EEG signal. The processing device may input the denoised EEG signal obtained by the denoising model into the discriminant model, and the discrimination result obtained by the discriminant model (which is referred to as a multiclassification result) may include a probability that the denoised EEG signal belongs to the plurality of types. The processing device may use a type with the highest probability as the classification result of the denoised EEG signal. When the discriminant model in the training of the denoising model based on the collected EEG signal is the multiclassification model, an optimization target for adjusting the denoising model based on the discrimination result may be to make the classification result of the denoised EEG signal outputted from the denoising model as clean as possible. The processing device may determine a plurality of losses regarding the plurality of types based on the discrimination result, and determine a loss function of the denoising model based on the plurality of losses. In some embodiments, the processing device may determine a weight corresponding to each loss regarding each type, determine a plurality of loss items based on the plurality of losses and the weights corresponding to the plurality of losses, and determine a loss function based on the plurality of loss items, and further adjust the model parameter of the denoising model based on the loss function. The loss regarding a certain type may be determined based on a difference between a probability corresponding to the type output by the discriminant model and a label value. The label value corresponding to the type of the EEG signal with the noises of the various types may be 0. The label value corresponding to the type of the clean EEG signal may be 1.

The target for adjusting the model parameter of the denoising model based on the loss function may include minimizing the value of each loss or loss item, i.e., minimizing the value of the loss or the loss item regarding the type of EEG signal with the noises of various types (e.g., the loss or the loss term regarding a type of EEG signal with various artifacts, the loss or the loss term regarding a type of EEG signal with environmental noise), and minimizing the value of the loss or the loss term regarding the type of clean EEG signal. Through this embodiment, it may be possible to realize a targeted adjustment of the model parameter of the denoising model to specifically improve the ability of the denoising model to remove a certain portion of noise. For example, if the discriminant model outputs a classification result as an EEG signal with eye movement artifacts, the model parameter of the denoising model may be adjusted in a targeted manner to improve the ability of the denoising model to remove the eye movement artifacts.

In some embodiments, the process of training the denoising model may further include training the discriminant model. The training the discriminant model may include inputting a plurality of denoised EEG signals and the clean EEG signal into an initial discriminant model, outputting the discrimination results corresponding to the denoised EEG signals and the clean EEG signal from the initial discriminant model, adjusting the model parameter of the initial discriminant model, so that the initial discriminant model is able to better distinguish between the denoised EEG signals and the clean EEG signal. As a result, the initial discriminant model may better discriminate whether the input signal is the clean EEG signal. In some embodiments, as described previously, the discriminant model may be the multiclassification model. In such cases, the model parameter of the discriminant model may be adjusted to enable the discriminant model to better differentiate between the plurality of types of EEG signal. The plurality of types may include EEG signals with the noises of various types (e.g., the EEG signal with eye movement artifacts, the EEG signal with muscle movement artifacts, the EEG signal with environmental noises, etc.) and the clean EEG signal.

Through multiple iterations of training, when the denoising model satisfies a condition (e.g., the model convergence), the trained denoising model may be obtained, which is the desired EEG signal denoising model corresponding to the EEG collection device. The EEG signal denoising model corresponding to the EEG collection device obtained by training may be used to remove the noise in the collected EEG signal, especially the noise brought by the EEG collection device itself, to obtain the clean EEG signal.

In some embodiments, the processing device may train the denoising model based on the collected EEG signal corresponding to a kind of the analog EEG signal. For example, the processing device may train the denoising model based on the collected EEG signal corresponding to the second analog EEG signal or the collected EEG signal corresponding to the third analog EEG signal.

In some embodiments, the processing device may train the denoising model based on the collected EEG signals corresponding to multiple kinds of analog EEG signals. For example, the processing device may train the denoising model based on the collected EEG signal corresponding to the first analog EEG signal, the collected EEG signal corresponding to the second analog EEG signal, and the collected EEG signal corresponding to the third analog EEG signal.

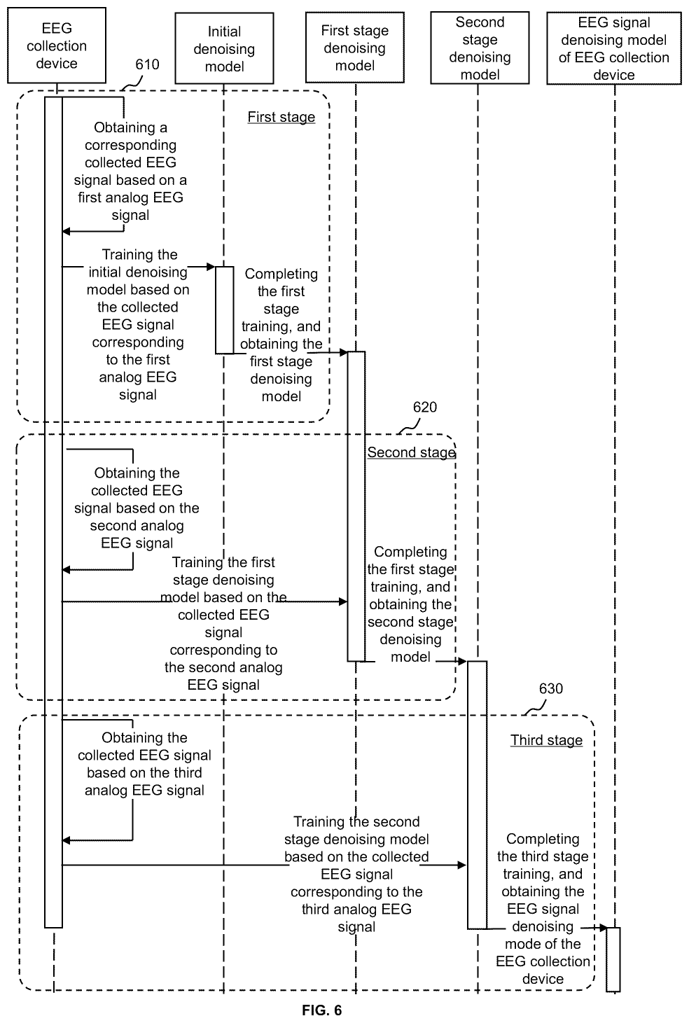

In some embodiments, the processing device may train the denoising model in stages. As the training stages progress, the processing device may gradually improve the noise reduction capability of the denoising model in a targeted manner. The processing device may train the denoising model in a plurality of stages based on the collected EEG signals corresponding to a plurality of analog EEG signals. More description regarding training the denoising model in the plurality of stages based on the collected EEG signals corresponding to the plurality of analog EEG signals may refer to FIG. 6 and the related descriptions, which is not be repeated here.

In some embodiments of the present disclosure, by using the EEG collection device to collect the analog EEG signal, the collected EEG signal may be obtained. Then a training on the denoising model may be performed by combining the discriminant model to obtain the denoising model with a strong denoising ability whose denoised EEG signal output is as clean as possible, which is the EEG signal denoising model of the EEG collection device, thereby realizing that the EEG signal denoising model of the EEG collection device obtained by training may remove the noise in the collected EEG signal, in particular, remove the noise brought by the EEG collection device itself.

FIG. 6 is a schematic diagram illustrating a method for training a denoising model in a staged manner according to some embodiments of the present disclosure.

In some embodiments, the processing device may perform a plurality of stages of training on the denoising model based on a collected EEG signal corresponding to a first analog EEG signal, a collected EEG signal corresponding to a second analog EEG signal, and a collected EEG signal corresponding to a third analog EEG signal, thereby obtaining the EEG signal denoising model corresponding to the EEG collection device. As shown in FIG. 6, the training of the denoising model may include a first stage 610, a second stage 620, and a third stage 630.

The first stage 610 of the training of the denoising model may include the following.

The processing device may train an initial denoising model based on the collected EEG signal corresponding to the first analog EEG signal. The initial denoising model refers to an untrained denoising model. After the initial denoising model is trained in the first stage, the denoising model obtained may be referred to as a first stage denoising model. The training of the initial denoising model in the first stage may be performed using the same process as the training of the denoising model described in step 430. More description regarding the training of the denoising model may refer to step 430 and the related descriptions. As mentioned above, the first analog EEG signal refers to a relatively regular or simple analog EEG signal that reflects an underlying feature of the EEG signal. It may be understood that the collected EEG signal corresponding to the first analog EEG signal does not have a high degree of difficulty in denoising, and accordingly, the first stage denoising model obtained by the training based on the collected EEG signal corresponding to the first analog EEG signal is able to denoise the relatively regular or simple collected EEG signal.

The second stage 620 of the training of the denoising model may include the following.

The first stage denoising model obtained in the first stage of training may be further trained based on the collected EEG signal corresponding to the second analog EEG signal. After the first stage denoising model is trained in the second stage, the obtained denoising model may be referred to as a second stage denoising model. The training of the first stage denoising model in the second stage may be performed using the same process as the training of the denoising model described in step 430. More description regarding the training of the denoising model may refer to step 430 and the related descriptions. As mentioned above, relative to the first analog EEG signal, the second analog EEG signal may be more complex and a change rule of the second analog EEG signal may be more in line with the analog EEG signal of the real EEG signal. It may be understood that the collected EEG signal corresponding to the second analog EEG signal is slightly more difficult for denoising than the collected EEG signal corresponding to the first analog EEG signal. The second stage denoising model trained based on the collected EEG signal corresponding to the second analog EEG signal may be able to perform the denoise on the collected EEG signal corresponding to the real EEG signal.

In some embodiments of the present disclosure, by performing the second stage training on the denoising model, the denoising model may learn about the real EEG signal, so that the denoising model may recognize the noise interspersed in the real EEG signal.

The third stage 630 of the training of the denoising model may include the following.

The second stage denoising model obtained in the second stage of training may be further trained based on the collected EEG signal corresponding to the third analog EEG signal. After the second stage denoising model is trained in the third stage, the EEG signal denoising model corresponding to the EEG collection device may be obtained. The training of the second stage denoising model in the third stage may be performed using the same process as the training of the denoising model described in step 430. More description regarding the training of the denoising model may refer to step 430 and the related descriptions. As mentioned above, the third analog EEG signal may be the EEG signal with other noises superimposed on the clean EEG signal, and the collected EEG signal corresponding to the third analog may be closer to the collected EEG signal of the actual situation. The EEG signal denoise model corresponding to the EEG collection device trained based on the collected EEG signal corresponding to the third analog EEG signal may have a stronger capability of denoising the collected EEG signal in the actual situation.

In some embodiments, the initial denoising model may also be referred to as an initial noise reducer. The first stage denoising model may also be referred to as a first stage denoiser, and the second stage noise model may also be referred to as a second stage denoiser.

In some embodiments of the present disclosure, by performing the first stage training on the denoising model, the denoising model may be made to learn a probability distribution of the noise brought by the operation of the EEG collection device itself, so that such noise may be removed by the first stage denoising model. A denoising capability of the second stage denoising model obtained in the second stage training may be stronger than the first stage denoising model, so that the second stage denoising model is able to remove the superimposed noise brought by the EEG collection device itself when collecting the real EEG signal. Through the third stage training, the EEG signal denoising model may be made more capable of denoising the collected EEG signal in real situations. The method of training the EEG signal denoising model by using different training samples in stages may make the EEG signal denoising model focus on how to denoise the EEG signals collected by the EEG collection device and to avoid an overfit of the EEG denoising model.

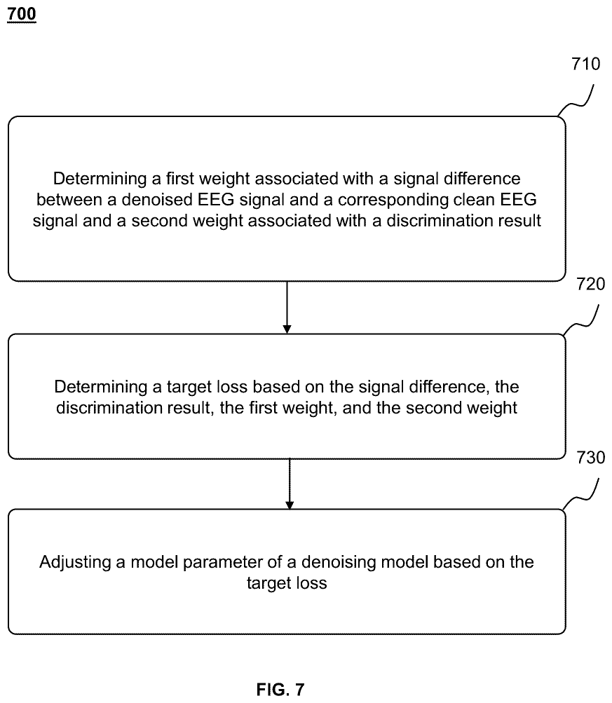

FIG. 7 is a flowchart illustrating an exemplary process for adjusting a model parameter of a denoising model according to some embodiments of the present disclosure. In some embodiments, a process 700 may be performed by a training module.

In some embodiments, each stage of the training of the denoising model may further include determining a signal difference between a denoised EEG signal output by the denoising model and a corresponding clean EEG signal, and adjusting the model parameter of the denoising model based on the signal difference and a discriminant result.

At the first stage 610, the processing device may process the collected EEG signal corresponding to the first analog EEG signal to obtain the corresponding denoised EEG signal based on the initial denoising model. The clean EEG signal corresponding to the denoised EEG signal obtained in the first stage 610 may be the first analog EEG signal. In such cases, the signal difference may be a signal difference between the denoised EEG signal obtained in the first stage 610 and the first analog EEG signal. In the second stage 620, the processing device may process the collected EEG signal corresponding to a second analog EEG signal to obtain the corresponding denoised EEG signal based on the first stage denoising model, and the clean EEG signal corresponding to the denoised EEG signal obtained in the second stage 620 may be the second analog EEG signal. In such cases, the signal difference may be a signal difference between the denoised EEG signal obtained in the second stage 620 and the second analog EEG signal. In the third stage 630, the processing device may process the collected EEG signal corresponding to a third analog EEG signal to obtain the corresponding denoised EEG signal based on the second stage denoising model, and the clean EEG signal corresponding to the denoised EEG signal obtained in the third stage 630 may be the clean EEG signal of the third analog EEG signal before the third analog EEG signal is superimposed with other noises. In such cases, the signal difference may be a signal difference between the denoised EEG signal obtained in the third stage 630 and the clean EEG signal before the third analog EEG signal is superimposed with the other noises.

As shown in FIG. 7, the adjusting the parameter of the denoising model based on the signal difference and the discriminant result may specifically include the following steps.

In step 710, a first weight associated with the signal difference between the denoised EEG signal and the corresponding clean EEG signal and a second weight associated with the discrimination result are determined.

The first weight may indicate an important degree of the signal difference for model optimization in the current training stage.

The second weight may indicate an important degree of the discrimination result for the model optimization in the current training stage.

In some embodiments, the first weight and the second weight may be preset. For different stages of the training of the denoising model, magnitudes of the first weight and the second weight may be different. As a result, in different training stages, the importance degrees of the signal differences and the discrimination results for the model optimization may be different.

As mentioned above, with the progression of the training stage, the denoising difficulty of the collected EEG signals may gradually increase, and the denoising ability of the trained denoising model may be gradually improved. Specifically, the ability of the denoising model may be improved from denoising a simple or regular collected EEG signal to denoising a complex collected EEG signal, and may further be improved to denoising the complex collected EEG signals with a variety of different noises. When training the denoising model with the ability to denoise the simple or regular collected EEG signal (i.e., the first training stage), a greater first weight and a smaller second weight may be set to make the model optimization focus more on the signal difference and less on the discrimination result. The denoising model obtained from training (the first stage denoising model) may output a denoised EEG signal that is as close as possible to the clean signal corresponding to the input signal. When the denoising model is trained to have the ability to denoise the complex collected EEG signal (i.e., the second stage), due to the increased complexity of the collected EEG signal, it is hoped that at this point, the denoising model may better learn and remove the distributions of more realistic and complex noises, and may no longer cause the denoising model to output the denoised EEG signal that is as close as possible to the corresponding clean signal. In such cases, it may be possible to reduce the first weight and increase the second weight based on the first stage, so that the second weight may be greater than the first weight, allowing the model optimization to focus more on the discrimination result and less on the signal difference. When the denoising model is trained to have the ability to noise reduction complex collected EEG signals with many different noises (i.e., the third stage), the first weight may be further reduced and the second weight may be further increased based on the second stage due to the further increase in the complexity of the collected EEG signal.

Exemplarily, the first weight and the second weight in the first stage may be preset to 0.6 and 0.4, respectively. The first weight and the second weight of the second stage may be preset to 0.3 and 0.7, respectively. The first weight and the second weight of the third stage may be preset to 0.1 and 0.9, respectively.

In step 720, a target loss is determined based on the signal difference, the discrimination result, the first weight, and the second weight.

In some embodiments, the target loss may be indicated as a joint loss function including a first loss item with respect to the signal difference and a second loss item with respect to the discrimination result. Exemplarily, the joint loss function may be indicated as:

$${Loss}_{c} = {X}_{N} * {Loss}_{A} + {Y}_{N} * {Loss}_{B}$$

where Loss_{A} denotes a first loss function with respect to the signal difference, Loss_{B} denotes a second loss function with respect to the discrimination result, Loss_{C} denotes the joint loss function, X_{N} denotes the first weight corresponding to the Nth stage of training, Y_{N} denotes the second weight corresponding to the Nth stage of training, X_{N}*Loss_{A} denotes the first loss item, and Y_{N}*Loss_{B} denotes the second loss item. N may be 1, 2, or 3.

In step 730, the model parameter of the denoising model is adjusted based on the target loss.

In some embodiments, a target of adjusting the model parameter of the denoising model based on the target loss may include minimizing the target loss. Specifically, the processing device may adjust the model parameter of the denoising model so that a value of the aforementioned first loss item may be minimized, and a value of the aforementioned second loss item may be minimized.

Through the method of training the denoising model in stages as described in some embodiments of the present disclosure, the weights corresponding to the signal difference and the discrimination result may be adjusted as the training stages change. For example, as the complexity of the analog EEG signal collected during the training process increases, the processing device may reduce the first weight of the signal difference and increase the second weight of the discrimination result. As a result, the denoising model may learn more about the clean EEG signal in an earlier stage to understand what the clean EEG signal is, and learn more about how to perform the denoising in a later training stage to recognize how to collect the EEG signal for denoising. In this manner, the denoising model may avoid overfitting based on the better learning of the clean EEG signal, and the generalization ability of the denoising model may be improved.

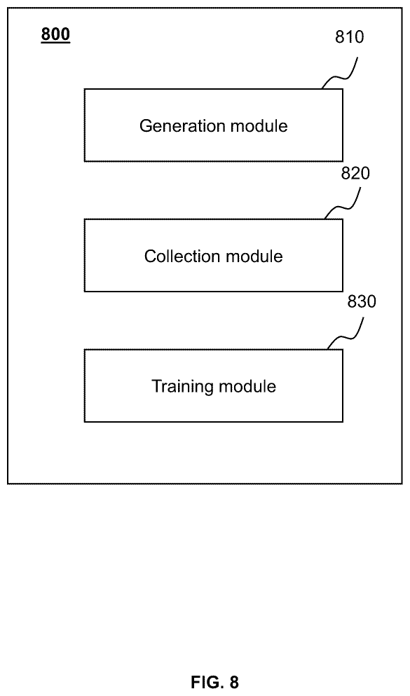

FIG. 8 is a module diagram illustrating a system for training an EEG signal denoising model of an EEG collection device according to some embodiments of the present disclosure.

In some embodiments, a system 800 for training the EEG signal denoising model of the EEG collection device may include a generation module 810, a collection module 820, and a training module 830.

The generation module 810 may be configured to generate an analog EEG signal. More description regarding the analog EEG signal may refer to FIG. 4 and the related descriptions, which are not repeated here. In some embodiments, the generation module 810 may be further configured to obtain a generation model. In some embodiments, the generation module 810 may be further configured to generate a second analog EEG signal based on the generation model. In some embodiments, the generation module 810 may be further configured to obtain the generation model by training a GAN. The generation model may be a generator in the GAN.

The collection module 820 may be configured to obtain a collected EEG signal by collecting the analog EEG signal by the EEG collection device. The collected EEG signal may contain noise caused by the EEG collection device. More description regarding the EEG collection device, the collected EEG signal, and the noise may refer to FIG. 4 and the related descriptions, which are not repeated here.

The training module 830 may be configured to obtain an EEG signal denoising model of the EEG collection device by training the denoising model based on the collected EEG signal. The training of the denoising model may include obtaining a denoised EEG signal by processing the collected EEG signal through the denoising model; obtaining a discrimination result by processing the denoised EEG signal through a discriminant model, the discrimination result reflecting a probability that the discriminant model determines that the processed denoised EEG signal is a clean EEG signal; and adjusting a model parameter of the denoising model based on the discrimination result. More description regarding the EEG signal denoising model, the denoised EEG signal, the discriminant model, the discrimination result, and the clean EEG signal may refer to FIG. 4 and the related descriptions, which are not repeated here. In some embodiments, when the analog EEG signal includes a first analog EEG signal, a second analog EEG signal, and a third analog EEG signal, the training module 830 may be configured to obtain the EEG signal denoising model of the EEG collection device by performing a plurality of stages of training on the denoising model based on the collected EEG signal corresponding to the first analog EEG signal, the collected EEG signal corresponding to the second analog EEG signal, and the collected EEG signal corresponding to the third analog EEG signal, respectively. More description regarding the first analog EEG signal, the second analog EEG signal, and the third analog EEG signal may refer to FIG. 4 and the related descriptions, which are not repeated here. More description regarding the plurality of stages of training may refer to FIG. 6 and the related descriptions, which are not repeated here. In some embodiments, the training module 830 may further be configured to obtain a first stage denoising model by performing a first stage training on an initial denoising model based on the collected EEG signal corresponding to the first analog EEG signal, obtain a second denoising model by performing a second stage training on the first stage denoising model based on the collected EEG signal corresponding to the second analog EEG signal, and obtain the EEG signal denoising model of the EEG collection device by perform a third stage training on the second stage denoising model based on the collected EEG signal corresponding to the third analog EEG signal. In some embodiments, the training module 830 may further be configured to determine a first weight associated with a signal difference between the denoised EEG signal and the corresponding clean EEG signal and a second weight associated with the discrimination result; determine a target loss based on the signal difference, the first weight, and the second weight; and adjust the model parameter of the denoising model based on the target loss. More description regarding the signal difference, the first weight, the second weight, and the target loss may refer to FIG. 7 and the related descriptions, which are not repeated here.

It may be understood that the above system and the modules thereof may be implemented in various manners.

It may be noted that the above descriptions of the system for training the EEG signal denoising model of the EEG collection device and the modules thereof are provided only for descriptive convenience, and do not limit the present disclosure to the scope of the embodiments. It may be understood that for those skilled in the art, after understanding the principle of the system, it may be possible to arbitrarily combine the individual modules or form a sub-system to be connected to the other modules without departing from this principle. In some embodiments, the modules in the system for training the EEG signal denoising model of the EEG collection device may be different modules in a single system, or a single module implementing the functions of two or more modules described above. For example, the modules may share a storage module, and the modules may each have a respective storage module. Deformations like these are within the scope of protection of the present disclosure.

Embodiments of the present disclosure further provide a system including at least one storage medium storing a training instruction set for the EEG signal denoising model of the EEG collection device and at least one processor in communication with the at least one storage medium. When performing the instruction set, the at least one processor is configured to generate the analog EEG signal; obtain the collected EEG signal by collecting the analog EEG signal through the EEG collection device, the collected EEG signal containing a noise caused by the EEG collection device; obtain the EEG signal denoising model of the EEG collection device by training a denoising model based on the collected EEG signal. The training of the denoising model may include obtaining a denoised EEG signal by processing the collected EEG signal through the denoising model; obtaining a discrimination result by processing the denoised EEG signal through a discriminant model, the discrimination result reflecting a probability that the discriminant model determines that the processed denoised EEG signal is a clean EEG signal; and adjusting a model parameter of the denoising model based on the discrimination result.

Embodiments of the present disclosure also provide a training device for an EEG signal denoising model of an EEG collection device, including at least one storage medium configured to store computer instructions and at least one processor configured to perform the computer instructions to implement the method of generating an analog EEG signal; obtaining a collected EEG signal by collecting the analog EEG signal through the EEG collection device, the collected EEG signal containing a noise caused by the EEG collection device; and obtaining the EEG signal denoising model of the EEG collection device by training a denoising model based on the collected EEG signal. The training of the denoising model includes obtaining a denoised EEG signal by processing the collected EEG signal through the denoising model; obtaining a discrimination result by processing the denoised EEG signal through a discriminant model, the discrimination result reflecting a probability that the discriminant model determines that the processed denoised EEG signal is a clean EEG signal; and adjusting a model parameter of the denoising model based on the discrimination result.

Embodiments of the present disclosure also provide a computer-readable storage medium storing computer instructions. When reading the computer instructions, a computer implements a method including generating an analog EEG signal; obtaining a collected EEG signal by collecting the analog EEG signal through the EEG collection device, the collected EEG signal containing a noise caused by the EEG collection device; and obtaining the EEG signal denoising model of the EEG collection device by training a denoising model based on the collected EEG signal. The training of the denoising model includes obtaining a denoised EEG signal by processing the collected EEG signal through the denoising model; obtaining a discrimination result by processing the denoised EEG signal through a discriminant model, the discrimination result reflecting a probability that the discriminant model determines that the processed denoised EEG signal is a clean EEG signal; and adjusting a model parameter of the denoising model based on the discrimination result.

The basic concepts have been described above, and it may be apparent to those skilled in the art that the foregoing detailed disclosure serves only as an example and does not constitute a limitation of the present disclosure. While not expressly stated herein, those skilled in the art may make various modifications, improvements, and amendments to the present disclosure. Those types of modifications, improvements, and amendments are suggested in the present disclosure, so they remain within the spirit and scope of the exemplary embodiments of the present disclosure.

Also, the present disclosure uses specific words to describe embodiments of the present disclosure. Such as “an embodiment,” “one embodiment,” and/or “some embodiments” means a feature, structure, or characteristic associated with at least one embodiment of the present disclosure. Accordingly, it may be emphasized and noted that the “one embodiment” or “an embodiment” or “an alternative embodiment” in different places in the present disclosure do not necessarily refer to the same embodiment. In addition, certain features, structures, or characteristics in one or more embodiments of the present disclosure may be suitably combined.

Furthermore, unless expressly stated in the claims, the order of the processing elements and sequences described herein, the use of numerical letters, or the use of other names are not intended to qualify the order of the processes and methods of the present disclosure. While some embodiments of the present disclosure that are currently considered useful are discussed in the foregoing disclosure by way of various examples, it may be appreciated that such details serve only illustrative purposes, and that additional claims are not limited to the disclosed embodiments, rather, the claims are intended to cover all amendments and equivalent combinations that are consistent with the substance and scope of the embodiments of the present disclosure. For example, although the implementation of various components described above may be embodied in a hardware device, it may also be implemented as a software only solution, e.g., an installation on an existing server or mobile device.

Similarly, it may be noted that in order to simplify the presentation of the disclosure of the present disclosure, and thereby aiding in the understanding of one or more embodiments of the present disclosure, the foregoing descriptions of embodiments of the present disclosure sometimes group multiple features together in a single embodiment, accompanying drawings, or in a description thereof. However, this method of disclosure does not imply that the objects of the present disclosure require more features than those mentioned in the claims. Rather, claimed subject matter may lie in less than all features of a single foregoing disclosed embodiment.

Some embodiments use numbers to describe the number of components, attributes, and it should be understood that such numbers used in the description of the embodiments are modified in some embodiments by the modifiers “about,” “approximately,” or “substantially.” Unless otherwise noted, the terms “about,” “approximate,” or “substantially” indicate that a ±20% variation in the stated number is allowed. Correspondingly, in some embodiments, the numerical parameters used in the present disclosure and the claims are approximations, which vary depending on the desired features of individual embodiments. In some embodiments, the numerical parameters may take into account the specified number of significant digits and employ general place-keeping. While the numerical domains and parameters used to confirm the breadth of their ranges in some embodiments of the present disclosure are approximations, in specific embodiments, such values are set to be as precise as possible within a feasible range.

For each of the patents, patent applications, patent application disclosures, and other materials cited in the present disclosure, such as articles, books, specification sheets, publications, documents, etc., the entire contents of which are hereby incorporated herein by reference. Application history documents that are inconsistent with or conflict with the contents of the present disclosure are excluded, as are documents (currently or hereafter appended to the present disclosure) that limit the broadest scope of the claims of the present disclosure. It should be noted that in the event of any inconsistency or conflict between the descriptions, definitions, and/or use of terms in the materials appended to the present disclosure and those set forth herein, the descriptions, definitions, and/or use of terms in the present disclosure shall prevail.

Finally, it may be understood that the embodiments described in the present disclosure are only used to illustrate the principles of the embodiments of the present disclosure. Other deformations may also fall within the scope of the present disclosure. As such, alternative configurations of embodiments of the present disclosure may be viewed as consistent with the teachings of the present disclosure as an example, not as a limitation. Correspondingly, the embodiments of the present disclosure are not limited to the embodiments expressly presented and described herein.

What is claimed is:

1. A method implemented on a computing device, the computing device including at least one processor and at least one storage medium storing a training instruction set for training an electroencephalogram (EEG) signal denoising model of an EEG collection device, the method comprising:
generating an analog EEG signal;
obtaining a collected EEG signal by collecting the analog EEG signal through the EEG collection device, the collected EEG signal containing a noise caused by the EEG collection device; and
obtaining the EEG signal denoising model of the EEG collection device by training a denoising model based on the collected EEG signal, the training of the denoising model comprising:
obtaining a denoised EEG signal by processing the collected EEG signal through the denoising model;
obtaining a discrimination result by processing the denoised EEG signal through a discriminant model, the discrimination result reflecting a probability that the discriminant model determines that the processed denoised EEG signal is a clean EEG signal; and
adjusting a model parameter of the denoising model based on the discrimination result.

2. The method of claim 1, wherein the analog EEG signal includes at least one of: a first analog EEG signal reflecting an underlying feature of the EEG signal, a second analog EEG signal simulating the clean EEG signal, and a third analog EEG signal with at least one other noise superimposed on the clean EEG signal.

3. The method of claim 2, the generating an analog EEG signal comprising:
obtaining a generation model; and
generating the second analog EEG signal based on the generation model.

4. The method of in claim 3, the obtaining a generation model comprising:
obtaining the generation model by training a generative adversarial network (GAN), wherein the generation model is a generator in the generative adversarial network.

5. The method of claim 2, wherein
the analog EEG signal includes the first analog EEG signal, the second analog EEG signal, and the third analog EEG signal, and
the obtaining the EEG signal denoising model of the EEG collection device by training a denoising model based on the collected EEG signal comprises:
obtaining the EEG signal denoising model of the EEG collection device by performing a plurality of stages of training on the denoising model based on the collected EEG signal corresponding to the first analog EEG signal, the collected EEG signal corresponding to the second analog EEG signal, and the collected EEG signal corresponding to the third analog EEG signal, respectively.

6. The method of claim 5, the obtaining the EEG signal denoising model of the EEG collection device by performing a plurality of stages of training on the denoising model based on the collected EEG signal corresponding to the first analog EEG signal, the collected EEG signal corresponding to the second analog EEG signal, and the collected EEG signal corresponding to the third analog EEG signal, respectively comprising:
obtaining a first stage denoising model by performing a first stage training on an initial denoising model based on the collected EEG signal corresponding to the first analog EEG signal;
obtaining a second stage denoising model by performing a second stage training on the first stage denoising model based on the collected EEG signal corresponding to the second analog EEG signal; and
obtaining the EEG signal denoising model of the EEG collection device by performing a third stage training on the second stage denoising model based on the collected EEG signal corresponding to the third analog EEG signal.

7. The method of claim 5, the adjusting a model parameter of the denoising model based on the discrimination result comprising:
in current training stage,
determining a first weight associated with a signal difference between the denoised EEG signal and the corresponding clean EEG signal and a second weight associated with the discrimination result;
determining a target loss based on the signal difference, the discrimination result, the first weight, and the second weight; and
adjusting the model parameter of the denoising model based on the target loss.

8. A system, comprising at least one storage medium storing a training instruction set for an EEG signal denoising model of an EEG collection device and at least one processor in communication with the at least one storage medium, wherein when performing the instruction set, the at least one processor is configured to:
generate an analog EEG signal;
obtain a collected EEG signal by collecting the analog EEG signal through the EEG collection device, the collected EEG signal containing a noise caused by the EEG collection device; and
obtain the EEG signal denoising model of the EEG collection device by training a denoising model based on the collected EEG signal, the training of the denoising model comprising:
obtaining a denoised EEG signal by processing the collected EEG signal through the denoising model;
obtaining a discrimination result by processing the denoised EEG signal through a discriminant model, the discrimination result reflecting a probability that the discriminant model determines that the processed denoised EEG signal is a clean EEG signal; and
adjusting a model parameter of the denoising model based on the discrimination result.

9. The system of claim 8, wherein the analog EEG signal includes at least one of: a first analog EEG signal reflecting an underlying feature of the EEG signal, a second analog EEG signal simulating the clean EEG signal, and a third analog EEG signal with at least one other noise superimposed on the clean EEG signal.

10. The system of in claim 8, wherein the at least one processor further is configured to:
obtain a generation model; and
generate the second analog EEG signal based on the generation model.

11. The system of in claim 10, wherein the at least one processor is further configured to:
obtain the generation model by training a generative adversarial network, wherein the generation model is a generator in the generative adversarial network.

12. The system of in claim 9, wherein
the analog EEG signal includes the first analog EEG signal, the second analog EEG signal, and the third analog EEG signal, and
the at least one processor is further configured to:
obtain the EEG denoising model of the EEG collection device by performing a plurality of stages of training on the denoising model based on the collected EEG signal corresponding to the first analog EEG signal, the collected EEG signal corresponding to the second analog EEG signal, and the collected EEG signal corresponding to the third analog EEG signal, respectively.

13. The system of in claim 12, wherein the at least one processor further configured to:
obtain a first stage denoising model by performing a first stage training on an initial denoising model based on the collected EEG signal corresponding to the first analog EEG signal;
obtain a second denoising model by performing a second stage training on the first stage denoising model based on the collected EEG signal corresponding to the second analog EEG signal; and
obtain the EEG signal denoising model of the EEG collection device by performing a third stage training on the second stage denoising model based on the collected EEG signal corresponding to the third analog EEG signal.

14. The system of in claim 12, wherein the at least one processor is further configured to:
determine a first weight associated with a signal difference between the denoised EEG signal and the corresponding clean EEG signal and a second weight associated with the discrimination result;
determine a target loss based on the signal difference, the first weight, and the second weight; and
adjust the model parameter of the denoising model based on the target loss.

15. A training device for an EEG signal denoising model of an EEG collection device, including at least one storage medium configured to store computer instructions and at least one processor configured to perform the computer instructions to implement the method of:
generating an analog EEG signal;
obtaining a collected EEG signal by collecting the analog EEG signal through the EEG collection device, the collected EEG signal containing a noise caused by the EEG collection device; and
obtaining the EEG signal denoising model of the EEG collection device by training a denoising model based on the collected EEG signal, the training of the denoising model comprising:
obtaining a denoised EEG signal by processing the collected EEG signal through the denoising model;
obtaining a discrimination result by processing the denoised EEG signal through a discriminant model, the discrimination result reflecting a probability that the discriminant model determines that the processed denoised EEG signal is a clean EEG signal; and
adjusting a model parameter of the denoising model based on the discrimination result.

16. The training device of claim 15, wherein the analog EEG signal includes at least one of: a first analog EEG signal reflecting an underlying feature of the EEG signal, a second analog EEG signal simulating the clean EEG signal, and a third analog EEG signal with at least one other noise superimposed on the clean EEG signal.

17. The training device of claim 16, the generating an analog EEG signal comprising:
obtaining a generation model; and
generating the second analog EEG signal based on the generation model.

18. The training device of in claim 17, the obtaining a generation model comprising:
obtaining the generation model by training a generative adversarial network (GAN), wherein the generation model is a generator in the generative adversarial network.

19. The training device of claim 16, wherein
the analog EEG signal includes the first analog EEG signal, the second analog EEG signal, and the third analog EEG signal, and
the obtaining the EEG signal denoising model of the EEG collection device by training a denoising model based on the collected EEG signal comprises:
obtaining the EEG signal denoising model of the EEG collection device by performing a plurality of stages of training on the denoising model based on the collected EEG signal corresponding to the first analog EEG signal, the collected EEG signal corresponding to the second analog EEG signal, and the collected EEG signal corresponding to the third analog EEG signal, respectively.

20. The training device of claim 19, the obtaining the EEG signal denoising model of the EEG collection device by performing a plurality of stages of training on the denoising model based on the collected EEG signal corresponding to the first analog EEG signal, the collected EEG signal corresponding to the second analog EEG signal, and the collected EEG signal corresponding to the third analog EEG signal, respectively comprising:
obtaining a first stage denoising model by performing a first stage training on an initial denoising model based on the collected EEG signal corresponding to the first analog EEG signal;
obtaining a second stage denoising model by performing a second stage training on the first stage denoising model based on the collected EEG signal corresponding to the second analog EEG signal; and
obtaining the EEG signal denoising model of the EEG collection device by performing a third stage training on the second stage denoising model based on the collected EEG signal corresponding to the third analog EEG signal.
